(19) United States
(12) Patent Application Publication
(10) Pub. No.: US 2025/0115857 A1
(43) Pub. Date: Apr. 10, 2025

(54) CELL CULTURING SYSTEM AND MOCK CARTRIDGE

(71) Applicants: Canon Kabushiki Kaisha, Tokyo (JP); CANON MEDICAL SYSTEMS CORPORATION, Tochigi (JP)

(72) Inventors: Kosuke KURACHI, Yokohama (JP); Makoto KOJIMA, Atsugi (JP)

(73) Assignees: Canon Kabushiki Kaisha, Tokyo (JP); CANON MEDICAL SYSTEMS CORPORATION, Tochigi (JP)

(21) Appl. No.: 18/907,150

(22) Filed: Oct. 4, 2024

(30) Foreign Application Priority Data
Oct. 6, 2023 (JP) 2023-174556

(51) Int. Cl. C12M 1/00; C12M 3/00

(52) U.S. Cl. CPC C12M 41/14; C12M 23/34; C12M 23/42

(57) ABSTRACT

A cell culturing system according to an embodiment includes an incubator and a mock cartridge. The incubator has a plurality of slots in each of which a cartridge including a culturing container for culturing cells may be installed and is configured to adjust a temperature to which the culturing containers in any of the installed cartridges are exposed. The mock cartridge is provided for a purpose of being installed into one or more, if any, of the plurality of slots having no cartridge installed therein and is configured to enclose therein no cells to be cultured.

### CROSS-REFERENCE TO RELATED APPLICATIONS

This application is based upon and claims the benefit of priority from Japanese Patent Application No. 2023-174556, filed on Oct. 6, 2023; the entire contents of which are incorporated herein by reference.

### FIELD

Embodiments described herein relate generally to a cell culturing system and a mock cartridge.

### BACKGROUND

To culture cells efficiently, a cell culturing system may have a plurality of slots in which a plurality of cartridges may be installed. In this situation, for example, each of the cartridges is a container including a culturing container used for culturing the cells. When a cell culturing system has a plurality of slots, it is necessary to control, with respect to a plurality of cartridges, the temperature to which the culturing containers in the cartridges are exposed, with a high level of precision. In such a cell culturing system, when the cartridges are installed only in a part of the slots so that all the slots do not have a cartridge installed, i.e., when there are one or more vacant slots, it may be difficult to exercise the temperature control with a high level of precision.

### BRIEF DESCRIPTION OF THE DRAWINGS

FIG. 1 is a drawing illustrating an exemplary configuration of a cell culturing system according to a first embodiment;

FIG. 2 is a diagram illustrating an exemplary functional configuration of the cell culturing system according to the first embodiment;

FIG. 3 is a drawing illustrating an exemplary configuration of an incubator according to the first embodiment;

FIG. 4 is a drawing for explaining an example of a procedure for installing a mock cartridge according to the first embodiment;

FIG. 5 is a diagram for explaining an example of a relationship between a thermal characteristic of cartridges and a thermal characteristic of a mock cartridge according to the first embodiment;

FIG. 6 is a drawing illustrating an example of an incubator in a cell culturing system according to a modification example of the first embodiment;

FIG. 7 is a drawing illustrating examples of mock cartridges according to a second embodiment;

FIG. 8 is a diagram illustrating an exemplary functional configuration of a cell culturing system according to the second embodiment; and

FIG. 9 is a flowchart illustrating an example of a flow in a process executed by a controlling function according to the second embodiment.

### DETAILED DESCRIPTION

One of the problems to be solved by the embodiments disclosed in the present specification and drawings is to exercise temperature control with a high level of precision. However, the problems to be solved by the embodiments disclosed in the present specification and drawings are not limited to the abovementioned problem. It is also possible to consider problems corresponding to advantageous effects achieved by any of the configurations described in the embodiments below as other problems.

A cell culturing system according to an embodiment includes an incubator and a mock cartridge. The incubator has a plurality of slots in each of which a cartridge including a culturing container for culturing cells may be installed and is configured to adjust a temperature to which the culturing containers in any of the installed cartridges are exposed. The mock cartridge is provided for a purpose of being installed into one or more, if any, of the plurality of slots having no cartridge installed therein and is configured to enclose therein no cells to be cultured.

Exemplary embodiments will be explained in detail below, with reference to the accompanying drawings. The following embodiments are not intended to limit the present disclosure set forth in the claims. Further, not all the combinations of features described in the present embodiments are necessarily requisite for the problem solving means of the present disclosure.

### First Embodiment

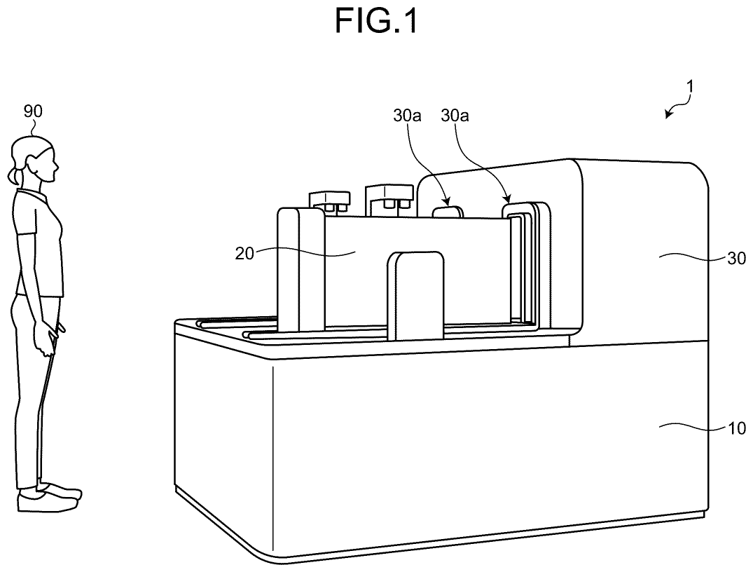

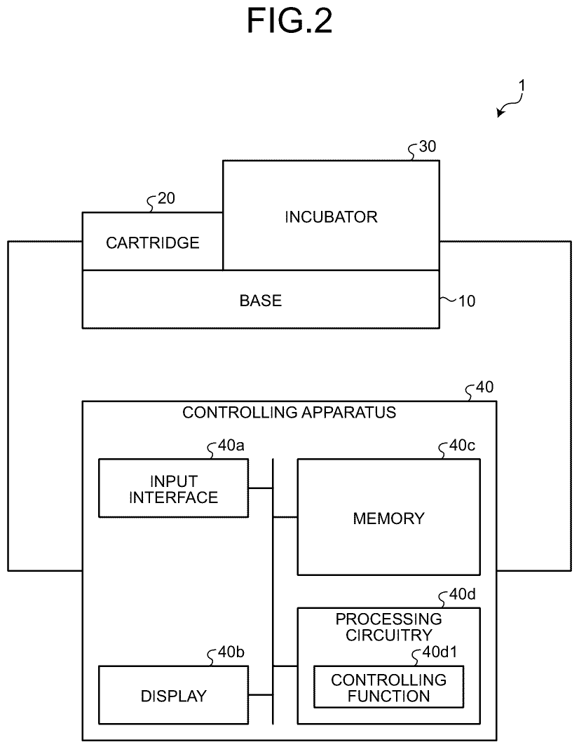

A cell culturing system according to a first embodiment will be explained. FIG. 1 is a drawing illustrating an exemplary external configuration of a cell culturing system 1 according to the first embodiment. As illustrated in FIG. 1, the cell culturing system 1 includes a base 10, a cartridge 20, and an incubator 30. Although not illustrated in FIG. 1, the cell culturing system 1 also includes a controlling apparatus 40 illustrated in FIG. 2. In the cell culturing system 1, the incubator 30 is configured to supply gas at a temperature suitable for culturing cells, to a culturing container in the cartridge 20, so that the cells in the culturing container are cultured.

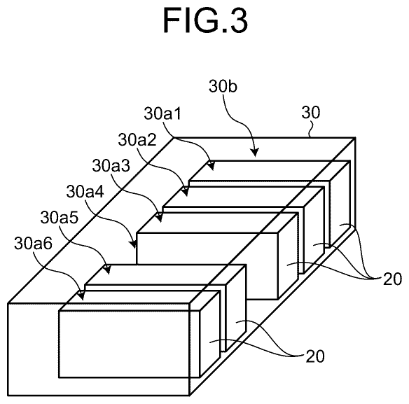

The base 10 is a base configured to support the incubator 30 while the cartridge 20 is placed thereon. The cartridge 20 placed on the base 10 is movable over the base 10. The cartridge 20 may be installed in one of the slots 30a as a result of being manually inserted into an internal space 30b (see FIG. 3) of the incubator 30 by a user 90 or may be installed in one of the slots 30a as a result of being automatically inserted into the internal space 30b of the incubator 30 by a moving mechanism (not illustrated). Details of the incubator 30 will be explained later.

The cartridge 20 is a container configured to store therein the culturing container containing the cells to be cultured. In other words, for example, the cartridge includes the culturing container used for culturing the cells. The cartridge 20 has a substantially cuboid shape, so that the culturing container is stored in an internal space of the cartridge 20. The culturing container is configured so that the cells in the culturing container are cultured by using a culture medium in the culturing container.

FIG. 2 is a diagram illustrating an exemplary functional configuration of the cell culturing system 1 according to the first embodiment. The controlling apparatus 40 illustrated in FIG. 2 is an apparatus configured to control the cartridge 20 installed in the slot 30a and the incubator 30. As illustrated in FIG. 2, the controlling apparatus 40 is electrically connected to the cartridge 20 installed in the slot 30a and to the incubator 30.

The controlling apparatus 40 includes an input interface 40a, a display 40b, a memory 40c, and processing circuitry 40d.

The input interface 40a is configured to receive various types of input operations from the user 90, to convert the received input operations into electrical signals, and to output the electrical signals to the processing circuitry 40d.

For example, the input interface 40a is realized by using a mouse, a keyboard, a trackball, a switch, a button, a joystick, a touchpad on which input operations can be performed by touching an operation surface thereof, a touch screen in which a display screen and a touchpad are integrally formed, contactless input circuitry using an optical sensor, audio input circuitry, and or the like. In this situation, the input interface 40a does not necessarily need to include physical operation component parts such as the mouse, the keyboard, and/or the like. For instance, possible examples of the input interface 40a include electrical signal processing circuitry configured to receive an electrical signal corresponding to an input operation from an external input mechanism provided separately from the controlling apparatus 40 and to further output the received electrical signal to the processing circuitry 40d.

The display 40b is configured to output various types of information by displaying the various types of information. For example, the display 40b is configured to output various types of information by displaying the various types of information generated by the processing circuitry 40d. For example, the display 40b may be a liquid crystal display or a Cathode Ray Tube (CRT) display. The display 40b may be of a desktop type or may be configured by using a tablet terminal or the like. The display 40b is an example of a display unit and is also an example of an output unit.

The memory 40c is realized by using, for example, a semiconductor memory element such as a Random Access Memory (RAM) or a flash memory, or a hard disk, an optical disc, or the like. For example, the memory 40c is configured to store therein various types of information generated by the processing circuitry 40d. Further, for example, the memory 40c is configured to store therein a program used by the processing circuitry 40d to realize various types of functions (e.g., a controlling function 40d1 explained later). Alternatively, the memory 40c may be realized by using a server group (a cloud) connected to the cell culturing system 1 via a network.

The processing circuitry 40d is configured to control operations of the entirety of the cell culturing system 1. For example, the processing circuitry 40d is configured to execute the controlling function 40d1. In this situation, for example, the controlling function 40d1 being a constituent element of the processing circuitry 40d illustrated in FIG. 1 is recorded within the memory 40c in the form of a computer-executable program. The processing circuitry 40d may be realized by using one or more processors, for example, and is configured to realize the controlling function 40d1 corresponding to the program, by reading the program from the memory 40c and executing the read program. In other words, the processing circuitry 40d that has read the program has the controlling function 40d1 illustrated within the processing circuitry 40d in FIG. 2.

The controlling function 40d1 is configured to exercise various types of control over the cartridge 20 installed in the slot 30a and the incubator 30. For example, on the basis of an instruction from the user 90 received via the input interface 40a, the controlling function 40d1 is configured to control the cartridge 20 so that various types of processes are performed on the culturing container in the cartridge 20. Further, on the basis of an instruction from the user 90 received via the input interface 40a, the controlling function 40d1 is configured to control the incubator 30 so as to adjust a temperature to which one or more culturing containers in one or more cartridges 20 installed in one or more slots 30a are exposed. In this situation, the “temperature to which the one or more culturing containers are exposed” denotes, more specifically, the “temperature of gas to which the one or more culturing containers are exposed”, for example.

Further, the term “processor” used in the above explanation denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). The one or more processors are configured to realize the function by reading the program saved in storage circuitry and executing the read program. Alternatively, instead of having the program saved in the storage circuitry, it is also acceptable to directly incorporate the program in the circuitry of the one or more processors. In that situation, the one or more processors are configured to realize the functions by reading the program incorporated in the circuitry thereof and executing the read program. Further, the processors of the present embodiments do not each necessarily need to be structured as a single piece of circuitry. It is also acceptable to structure one processor by combining together a plurality of pieces of independent circuitry so as to realize the functions thereof.

FIG. 3 is a drawing illustrating an exemplary configuration of an incubator 30 according to the first embodiment. The example in FIG. 3 schematically illustrates the incubator 30. As illustrated in FIG. 3, the incubator 30 includes a plurality of (six) slots 30a1 to 30a6. In each of the plurality of slots 30a1 to 30a6, it is possible to install a cartridge 20. In the following sections, when being distinguished from one another, the slots will be referred to as the slots 30a1 to 30a6. When not being distinguished from one another, the plurality of slots may be referred to as the slots 30a. In the following sections, an example will be explained in which the quantity of the slots 30a included in the incubator 30 is six. However, the quantity of the slots 30a do not necessarily need to be six and may be any quantity that is two or larger.

To ensure that the temperature to which the one or more culturing containers in the one or more cartridges 20 installed in the incubator 30 are exposed is a temperature (a goal temperature) suitable for culturing the cells, the incubator 30 is placed under the abovementioned control exercised by the controlling function 40d1, while the incubator 30 is configured to adjust the temperature to which the culturing containers in the installed cartridges 20 are exposed. More specifically, the incubator 30 has the internal space 30b and is configured to indirectly adjust the temperature to which the culturing containers are exposed, by directly adjusting the temperature of the gas in the internal space 30b.

In this situation, in the incubator 30 according to the present embodiment, each of the slots 30a is provided with one insertion opening into which a cartridge 20 may be inserted. In the present embodiment, the plurality of slots 30a are provided, and in correspondence therewith, a plurality of insertion openings are provided. In contrast, the internal space 30b is a space shared by all the (plurality of) slots 30a. In other words, while the quantity of the slots 30a and the quantity of the insertion openings are each two or more, the quantity of the internal space 30b is one.

In the incubator 30 configured as described above, when there are one or more vacant slots, it may be difficult to exercise the temperature control with a high level of precision. An example of the reasons will be explained. For instance, in the example illustrated in FIG. 3, when there is no vacant slot, i.e., when a cartridge 20 is installed in each of all the slots 30a1 to 30a6, each of the four cartridges 20 installed in the slots 30a2 to 30a5 is impacted by heat generated by the adjacent two cartridges 20 positioned on either side thereof. All the cartridges 20 have an identical or substantially identical thermal characteristic. For this reason, the four cartridges 20 installed in the slots 30a2 to 30a5 are impacted by the heat equally or substantially equally.

In contrast, although each of the two cartridges 20 installed in the slots 30a1 and 30a6 positioned at the two ends is impacted by heat generated by the one adjacent cartridge 20 positioned on one side thereof, no cartridge 20 is present on the other side. For this reason, the two cartridges 20 installed in the slots 30a1 and 30a6 are impacted by the heat equally or substantially equally.

Consequently, although the impact of the heat exerted on the four cartridges 20 installed in the slots 30a2 to 30a5 is different from the impact of the heat exerted on the two cartridges 20 installed in the slots 30a1 and 30a6, the impact of the heat exerted on the four cartridges 20, which constitute majority of the six cartridges 20, is equal or substantially equal. Further, when there is no vacant slot, the number of types of impact of the heat exerted on the cartridges 20 is at most two. For these reasons, when there is no vacant slot, the incubator 30 is capable of relatively easily exercising the temperature control with a high level of precision.

On the contrary, when there is a vacant slot, as illustrated in FIG. 3, more specifically, when no cartridge 20 is installed in the slot 30a4, the one cartridge 20 installed in the slot 30a2 is impacted by heat generated by the two adjacent cartridges 20 positioned on either side thereof.

Further, each of the two cartridges 20 installed in the slots 30a3 and 30a5 is impacted by heat generated by the one adjacent cartridge 20 positioned on the one side thereof and is also impacted by heat generated by the one cartridge 20 positioned on the other side at an interval corresponding to one cartridge 20. For this reason, the two cartridges 20 installed in the slots 30a3 and 30a5 are impacted by the heat equally or substantially equally.

The impact of the heat exerted on each of the two cartridges 20 installed in the slots 30a1 and 30a6 positioned at the two ends is the same as that in the situation when there is no vacant slot.

Consequently, when there is a vacant slot as illustrated in FIG. 3, the impact of the heat exerted on the one cartridge 20 installed in the slot 30a2, the impact of the heat exerted on the two cartridges 20 installed in the slots 30a3 and 30a5, and the impact of the heat exerted on the two cartridges 20 installed in the slots 30a1 and 30a6 are all different from one another. For this reason, when there is a vacant slot as illustrated in FIG. 3, the number of types of impact of the heat exerted on the cartridges 20 are as many as three. It would therefore be difficult for the incubator 30 to exercise the temperature control with a high level of precision.

Further, for example, in the example illustrated in FIG. 3, because it would be difficult to exercise the temperature control with a high level of precision, there would be a relatively large temperature variation. In this situation, the temperature variation is an index value obtained by dividing the sum of differences between each of a plurality of temperatures of the plurality of culturing containers in the plurality of installed cartridges 20 and the goal temperature, by the quantity of the plurality of installed cartridges 20. For example, in the example of FIG. 3, the temperature variation is an index value obtained by dividing the sum of the differences between each of the five temperatures of the five culturing containers in the five installed cartridges 20 and the goal temperature, by the quantity of the cartridges 20 being “5”.

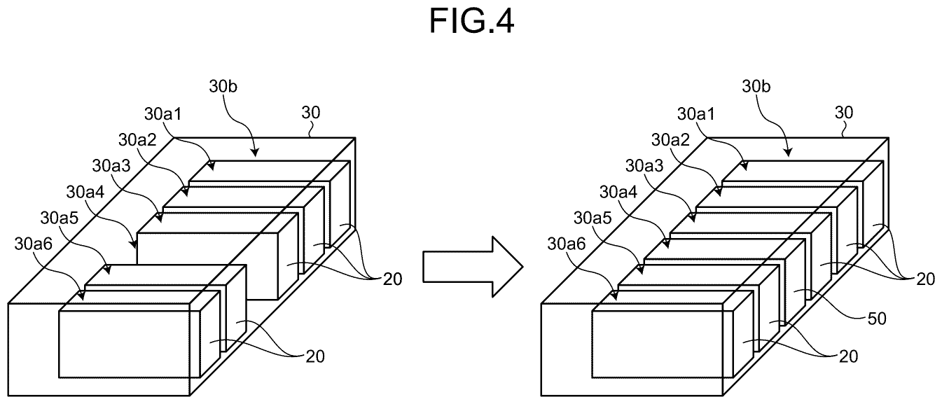

In the circumstances described above, to make it possible to exercise the temperature control with a high level of precision, the cell culturing system 1 according to the first embodiment includes at least one mock cartridge (dummy cartridge) 50. Further, as illustrated in the right section of FIG. 4, in the cell culturing system 1, the mock cartridge 50 is installed in the slot 30a4. In this situation, FIG. 4 is a drawing for explaining an example of a procedure for installing the mock cartridge 50 according to the first embodiment. The mock cartridge 50 is provided for the purpose of being installed in one or more slots 30a, if any, having no cartridge 20 installed therein among the plurality of slots 30a and is a cartridge that is mock and is configured to enclose therein no cells to be cultured. To make it possible to install the mock cartridge 50 in any of the slots 30a similarly to the cartridges 20, the shape of the mock cartridge 50 is identical or substantially identical to the shape of each of the cartridges 20.

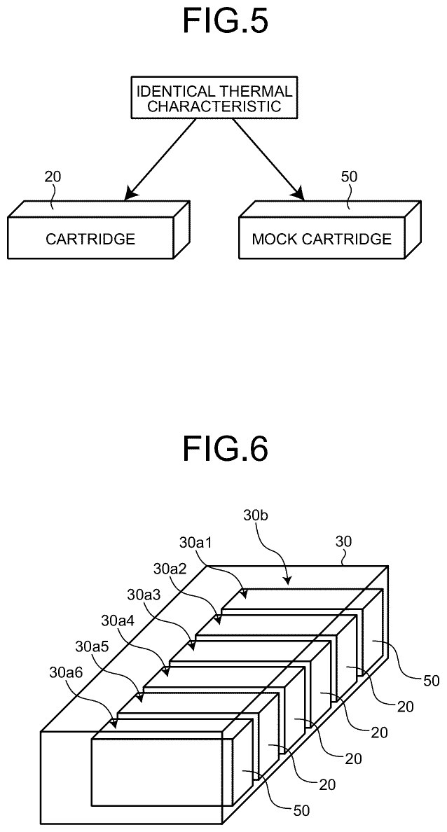

Further, as illustrated in FIG. 5, a thermal characteristic of the mock cartridge 50 is identical to a thermal characteristic of the cartridges 20. FIG. 5 is a diagram for explaining an example of a relationship between the thermal characteristic of the cartridges 20 and the thermal characteristic of the mock cartridge 50 according to the first embodiment. For example, the thermal characteristic is at least one of characteristics such as specific heat, a heat capacity, heat conductivity, heat diffusion, heat resistance, a coefficient of thermal expansion, and/or thermal strain that are determined by the size of the cartridges 20, the material of casings of the cartridges 20, and/or the like. The thermal characteristic of the mock cartridge 50 may be substantially identical to the thermal characteristic of the cartridges 20.

As explained above, in the cell culturing system 1 according to the first embodiment, the mock cartridge 50 is installed in each of all the slots 30a having no cartridge 20 installed therein, so that the cartridges having identical or substantially identical thermal characteristic are installed in all the slots 30a. With this configuration, the cell culturing system 1 is capable of exercising the temperature control with a high level of precision. Further, as a result, the cell culturing system 1 is able to prevent the occurrence of a large temperature variation.

Further, it is desirable when the quantity of the mock cartridges 50 is equal to the quantity of the plurality slots 30a included in the incubator 30 or is equal to or larger than the quantity of the plurality of slots 30a included in the incubator 30. The reason is that, when gas is to be eliminated from the cell culturing system 1, the mock cartridge 50 will need to be installed in each of all the slots 30a in the incubator 30.

The cell culturing system 1 according to the first embodiment has thus been explained. The cell culturing system 1 according to the embodiment is capable to exercising the temperature control with a high level of precision, as explained above.

### A Modification Example of First Embodiment

Next, the cell culturing system 1 according to a modification example of the first embodiment will be explained. In the following description of the modification example, differences from the cell culturing system 1 according to the first embodiment described above will primarily be explained, and explanations of the same configurations may be omitted.

For example, let us discuss an example in which a cartridge 20 is installed in each of all the slots 30a1 to 30a6. In that situation, as explained above, the impact of the heat exerted on the two cartridges 20 installed in the slots 30a1 and 30a6 positioned at the two ends is different from the impact of the heat exerted on the other cartridges 20 (i.e., the four cartridges installed in the slots 30a2 to 30a5). For this reason, to begin with, the temperature to which the culturing containers in the two cartridges 20 installed in the slots 30a1 and 30a6 positioned at the two ends are exposed is relatively greatly different from the temperature to which the culturing containers in the other cartridges 20 are exposed. For example, there is a difference equal to or larger than a prescribed value between an average value of the six temperatures to which the six culturing containers in the six cartridges 20 installed in the slots 30a1 to 30a6 are exposed and the temperature to which each of the two culturing containers in the two cartridges 20 installed in the slots 30a1 and 30a6 positioned at the two ends is exposed.

As explained above, when the plurality of cartridges 20 are installed in the plurality of slots 30a1 to 30a6, respectively, the slots 30a1 and 30a6 positioned at the two ends are the slots having installed therein the two cartridges 20 including the culturing containers exposed to a specific temperature. In this situation, the specific temperature denotes a temperature of which the difference from the abovementioned average value is equal to or larger than the prescribed value. The prescribed value may be in the range of 0.2 degrees to 1 degree, for example.

FIG. 6 is a drawing illustrating an example of an incubator 30 in the cell culturing system 1 according to the modification example of the first embodiment. In the present modification example, as illustrated in FIG. 6, a mock cartridge 50 is installed in each of the slots 30a1 and 30a6 positioned at the two ends. Further, a cartridge 20 is installed in at least one of the remaining slots 30a2 to 30a5. Accordingly, by using the cell culturing system 1 according to the modification example of the first embodiment, it is possible to install the one or more cartridges 20 while avoiding the slots 30a1 and 30a6 which are positioned at the two ends and in which, to begin with, the culturing containers are exposed to the specific temperature. Consequently, the cell culturing system 1 according to the modification example of the first embodiment is able to further prevent the occurrence of a large temperature variation.

### Second Embodiment

Next, the cell culturing system 1 according to a second embodiment will be explained. In the following description of the second embodiment, differences from the cell culturing system 1 according to the first embodiment and the modification example described above will primarily be explained, and explanations of the same configurations may be omitted.

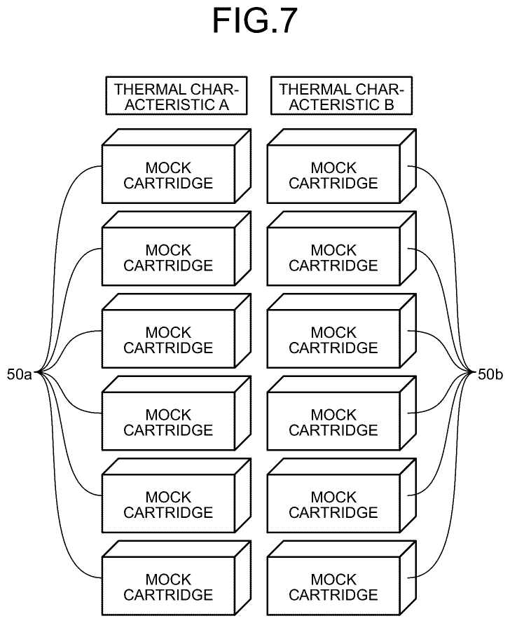

FIG. 7 is a drawing illustrating examples of mock cartridges 50a and 50b according to the second embodiment. As illustrated in FIG. 7, the cell culturing system 1 according to the second embodiment includes the plurality of mock cartridges 50a and 50b having mutually-different thermal characteristics. For example, the six mock cartridges 50a illustrated in FIG. 7 have thermal characteristic A, whereas the six mock cartridges 50b have thermal characteristic B, which is different from thermal characteristic A. For example, it is acceptable to ensure that the thermal characteristic of the mock cartridges 50a is different from the thermal characteristic of the mock cartridges 50b, by making the size of the casings of the mock cartridges 50a different from the size of the casings of the mock cartridges 50b. Alternatively, it is also acceptable to ensure that the thermal characteristic of the mock cartridges 50a is different from the thermal characteristic of the mock cartridges 50b, by making a material of the casings of the mock cartridges 50a different from a material of the casings of the mock cartridges 50b. Furthermore, the cell culturing system 1 is not limited to including the mock cartridges 50a and 50b having the two types of thermal characteristics and may include mock cartridges having three or more types of thermal characteristics.

Similarly to the quantity of the mock cartridges 50 according to the first embodiment, it is desirable when the quantity of the mock cartridges 50a is equal to the quantity of the plurality of slots 30a included in the incubator 30 or is equal to or larger than the quantity of the plurality of slots 30a included in the incubator 30. Additionally, it is also desirable when the quantity of the mock cartridges 50b is equal to the quantity of the plurality of slots 30a included in the incubator 30 or is equal to or larger than the quantity of the plurality of slots 30a included in the incubator 30.

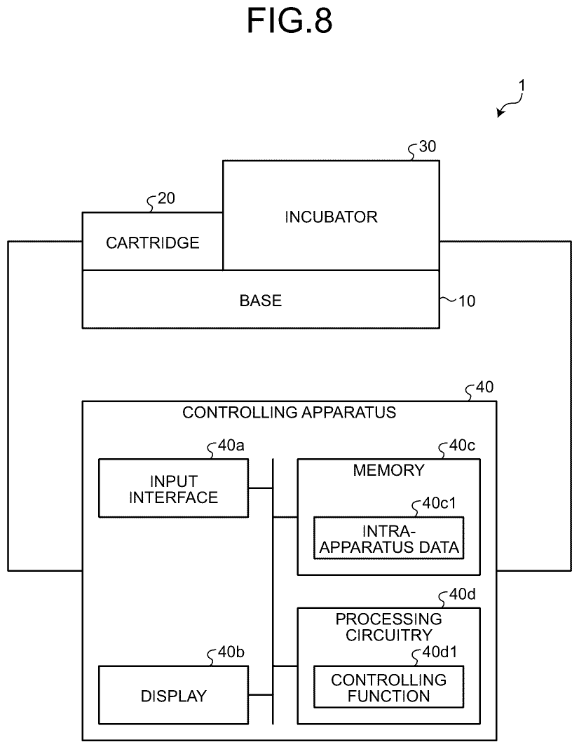

FIG. 8 is a diagram illustrating an exemplary functional configuration of the cell culturing system 1 according to the second embodiment. As illustrated in FIG. 8, the controlling apparatus 40 according to the second embodiment is different from the controlling apparatus 40 according to the first embodiment in that the memory 40c has stored therein intra-apparatus data 40c1.

The intra-apparatus data 40c1 is data (a table) having registered therein records in which all the possible combinations of the cartridges (the cartridges 20 and the mock cartridges 50a and 50b) that may be installed in the plurality of slots 30a1 and 30a6 are kept in correspondence with temperature variations for those combinations, the records being registered in a quantity equal to the quantity of all the possible combinations. In other words, there are three possibilities where a cartridge 20 is installed in each of the plurality of slots 30a1 to 30a6, where at least one mock cartridge 50a is installed, and where at least one mock cartridge 50b is installed. Consequently, the intra-apparatus data 40c1 has registered therein eighteen records, which is calculated as 6 (the slots 30a1 to 30a6)×3 (the three possible situations).

The temperature variations registered in the intra-apparatus data 40c1 are obtained through an experiment or a simulation.

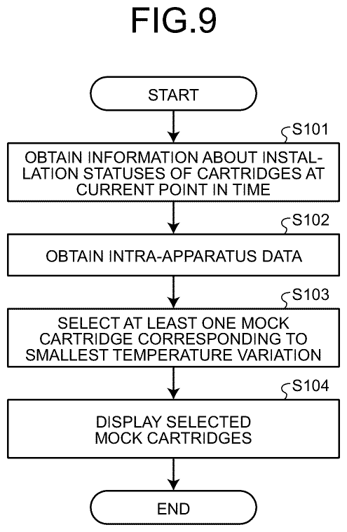

FIG. 9 is a flowchart illustrating an example of a flow in a process executed by the controlling function 40d1 according to the second embodiment. As illustrated in FIG. 9, to begin with, the controlling function 40d1 obtains information about installation statues of the cartridges 20 at a current point in time (step S101). The information about the installation statuses of the cartridges 20 denotes, for example, information indicating which of the slots 30a1 to 30a6 has a cartridge 20 installed therein.

For example, at step S101, the controlling function 40d1 causes the display 40b to display a screen for prompting the user 90 to input the information about the installation statuses of the cartridges 20 at the current point in time. Further, the controlling function 40d1 obtains the information about the installation statuses of the cartridges 20 at the current point in time which was input by the user 90 via the input interface 40a.

Alternatively, when the incubator 30 holds the information about the installation statuses of the cartridges 20 at the current point in time, the controlling function 40d1 may obtain, from the incubator 30, the information about the installation statuses of the cartridges 20 at the current point in time.

After that, the controlling function 40d1 obtains the intra-apparatus data 40c1 (step S102). The obtained intra-apparatus data 40c1 is to be used in a process performed at step S103.

Subsequently, the controlling function 40d1 selects, from within the intra-apparatus data 40c1, at least one mock cartridge that matches the information about the installation statuses of the cartridges 20 at the current point in time and that corresponds to a situation having the smallest temperature variation (step S103).

A process at step S103 will be explained. For example, from among all the records registered in the intra-apparatus data 40c1 the controlling function 40d1 specifies a plurality of records that match the information about the installation statues of the cartridges 20 at the current point in time which was obtained at step S101. In this situation, an example will be explained in which the information about the installation statuses of the cartridges 20 at the current point in time, which was obtained at step S101, indicates that a cartridge 20 is installed in each of the slots 30a1, 30a3, and 30a5. In this situation, at step S103, from among all the records registered in the intra-apparatus data 40c1, the controlling function 40d1 specifies the plurality of records indicating that a cartridge 20 is installed in each of the slots 30a1, 30a3, and 30a5.

After that, from among the plurality of specified records, the controlling function 40d1 specifies one of the registered records corresponding to the smallest temperature variation. Subsequently, the controlling function 40d1 selects the mock cartridges indicated in the one specified record as being installed in the slots 30a2, 30a4, and 30a6 that are other than the slots 30a1, 30a3, and 30a5.

In this manner, at step S103, from among the plurality of mock cartridges (the mock cartridges 50a and 50b), the controlling function 40d1 selects the mock cartridges to be installed in the slots 30a each having no cartridge 20 installed therein, in accordance with the information about the cartridges 20 installed in the slots 30a.

After that, the controlling function 40d1 causes the display 40b to display information indicating the selected mock cartridges (e.g., information indicating the types of the mock cartridges) (step S104). In other words, the controlling function 40d1 is configured to cause the display 40b to output the information indicating the selected mock cartridges. Alternatively, at step S104, the controlling function 40d1 may cause an audio output apparatus configured to output audio to output audio indicating the selected mock cartridges. After that, the controlling function 40d1 ends the process illustrated in FIG. 9.

As a result of the process illustrated in FIG. 9, it is possible to assist the user 90 in understanding the mock cartridges to be installed into the slots 30a each having no cartridge 20 installed therein. Accordingly, the user 90 is able to install the appropriate mock cartridges into the slots 30a each having no cartridge 20 installed therein.

### A Modification Example of Second Embodiment

In the second embodiment above, the example was explained in which the single type of cartridges 20 was used. However, the cell culturing system 1 may include a plurality of types of cartridges 20 that use mutually-different culturing conditions at the time of culturing the cells. Thus, this modification example will be explained as a modification example of the second embodiment. In the following description of the modification example of the second embodiment, differences from the cell culturing system 1 according to the second embodiment described above will primarily be explained, and explanations of the same configurations may be omitted.

In the modification example of the second embodiment, at step S101, as the information about the installation statuses of the cartridges 20 at the current point in time, the controlling function 40d1 obtains, for example, information indicating which of the slots 30a1 to 30a6 has which type of cartridge 20 installed therein.

Subsequently, at step S102, the controlling function 40d1 obtains the intra-apparatus data 40c1. The intra-apparatus data 40c1 in the modification example of the second embodiment is data (a table) having registered therein records in which all the possible combinations of the cartridges (the cartridges 20 and the mock cartridges 50a and 50b) that may be installed in the plurality of slots 30a1 and 30a6 are kept in correspondence with temperature variations for those combinations, the records being registered in a quantity equal to the quantity of all the possible combinations. In this situation, when a cartridge 20 is installed in any of the slots 30a, the record thereof also includes the type of the cartridge 20.

After that, at step S103, from within the intra-apparatus data 40c1, the controlling function 40d1 selects at least one mock cartridge that matches the information about the installation statues of the cartridges 20 at the current point in time and that corresponds to a situation having the smallest temperature variation.

Subsequently, at step S104, the controlling function 40d1 causes either the display 40b or an audio output apparatus to output information indicating the selected mock cartridge. Accordingly, similarly to the second embodiment, the user 90 is able to install the appropriate mock cartridges into the slots 30a each having no cartridge 20 installed therein.

In the embodiments and the modification examples described above, the example was explained in which the internal space 30b of the incubator 30 is shared among all the slots 30a. However, it is also possible to apply any of the techniques according to the embodiments and the modification examples described above to a situation where there are a plurality of internal spaces, while slots 30a in a prescribed quantity are regarded as one block so that the internal spaces 30b are divided by component parts in correspondence with the blocks, as long as the component parts have relatively low thermal insulation property, and heat is conducted relatively easily between adjacently-positioned internal spaces.

According to at least one aspect of the embodiments and the modification examples described above, it is possible to exercise the temperature control with a high level of precision.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell culturing system comprising:
an incubator that has a plurality of slots in each of which a cartridge including a culturing container for culturing cells may be installed and that is configured to adjust a temperature to which the culturing containers in any of the installed cartridges are exposed; and
a mock cartridge provided for a purpose of being installed into one or more, if any, of the plurality of slots having no cartridge installed therein and configured to enclose therein no cells to be cultured.

2. The cell culturing system according to claim 1, wherein the incubator has an internal space shared among the plurality of slots and is configured to adjust the temperature to which the culturing containers are exposed, by adjusting a temperature of gas in the internal space.

3. The cell culturing system according to claim 1, wherein a thermal characteristic of the mock cartridge is identical to a thermal characteristic of the cartridges.

4. The cell culturing system according to claim 1, wherein, when a plurality of cartridges are installed in the plurality of slots, respectively, the mock cartridge is to be installed into any of the slots having installed therein a cartridge including a culturing container exposed to a specific temperature.

5. The cell culturing system according to claim 4, wherein, among the plurality of slots, the mock cartridge is to be installed into each of slots at two ends, which serve as the slots each having installed therein the cartridge including the culturing container exposed to the specific temperature.

6. The cell culturing system according to claim 4, wherein, when the plurality of cartridges are installed in the plurality of slots, respectively, the mock cartridge is to be installed in any of the slots having installed therein the cartridge including the culturing container exposed to the specific temperature, being a temperature different, by a prescribed value or larger, from an average value of a plurality of temperatures to which the plurality of culturing containers in the plurality of installed cartridges are exposed.

7. The cell culturing system according to claim 1, comprising a plurality of mock cartridges including the mock cartridge and having mutually-different thermal characteristics.

8. The cell culturing system according to claim 7, comprising a controlling unit configured to select, from among the plurality of mock cartridges, a mock cartridge to be installed into any of the slots having no cartridge installed therein, in accordance with information about the cartridges installed in the slots, the controlling unit being configured to cause an output unit to output the selected mock cartridge.

9. A mock cartridge that is provided for a purpose of being installed into one or more, if any, of a plurality of slots of an incubator having no cartridge installed therein and that is configured to enclose therein no cells to be cultured, the incubator having the plurality of slots in each of which a cartridge including a culturing container for culturing cells may be installed and being configured to adjust a temperature to which the culturing containers in any of the installed cartridges are exposed.
